(19) United States
(12) Patent Application Publication
(10) Pub. No.: US 2011/0288234 A1
(43) Pub. Date: Nov. 24, 2011

(54) SILICA NANOPARTICLES POSTLOADED WITH PHOTOSENSITIZERS FOR DRUG DELIVERY IN PHOTODYNAMIC THERAPY

(75) Inventors: Ravindra K. Pandey, Williamsville, NY (US); Lalit N. Goswami, Columbia, MO (US); Allan Oseroff, Buffalo, NY (US); Stephanie Pincus, Buffalo, NY (US); Janet Morgan, Buffalo, NY (US); Paras N. Prasad, Williamsville, NY (US); Earl J. Bergey, South Dayton, NY (US)

(73) Assignees: The Research Foundation on State University of NY, Amherst, NY (US); Health Research, Inc., Buffalo, NY (US)

(21) Appl. No.: 12/918,232

(22) PCT Filed: Feb. 19, 2009
(86) PCT No.: PCT/US09/01029

§ 371 Date: Nov. 22, 2010

Related U.S. Application Data
(60) Provisional application No. 61/066,304, filed on Feb. 19, 2008.

(51) Int. Cl. C08G 77/38; C08F 130/08; C08G 77/388; B82Y 5/00

(52) U.S. Cl. 525/54.1; 525/474; 525/54.2; 525/326.5; 977/773; 977/788; 977/906; 977/927; 977/897; 977/930; 977/928

(57) ABSTRACT

A nanoparticle including a polysiloxane base having an exterior surface and having a photosensitizer at least partly exposed at its exterior surface, said photosensitizer being secured to the exterior surface by loading the photosensitizer onto the surface after formation of the polysiloxane base of the nanoparticle. The nanoparticle may have tumor targeting moieties and may be post loaded with cyanine dye. The nanoparticle preferably includes post loaded moieties providing at least two of tumor specificity, photodynamic properties and imaging capabilities and the photosensitizer is tagged with a radioisotope. A method for preparation of the nanoparticle is also provided.

### CROSS REFERENCE TO RELATED APPLICATION

This application claims priority from U.S. Provisional Application Ser. No. 61/066,304 filed Feb. 19, 2008 and is the National Stage of International Application No. PCT/US2009/001029, filed Feb. 19, 2009.

### STATEMENT REGARDING FEDERALLY SPONSORED RESEARCH OR DEVELOPMENT

This work was supported by grants form the National Institute of Health (R01CA119358-01, and RO1CA104492, 1R21CA109914). The United States Government may have certain rights in this invention.

### BACKGROUND OF THE INVENTION

The present invention relates to the field of nanoparticle mediated drug delivery in photodynamic therapy.

Photodynamic therapy (PDT), a light-activated treatment for cancer and other diseases, has emerged as one of the important areas in biophotonics research. PDT utilizes light-sensitive drugs or photosensitizers (PS), which are preferentially localized in malignant tissues upon systemic administration. The therapeutic effect is activated by the photoexcitation of the localized photosensitizers and the subsequent generation of cytotoxic species, such as singlet oxygen (1O2), free radicals or peroxides, which lead to selective and irreversible destruction of the diseased tissues without damaging adjacent healthy ones.

In spite of the advantages over current treatments including surgery, radiation therapy and chemotherapy, PDT still has problems to be resolved for a more general clinical acceptance. One of the major challenges in PDT is the preparation of stable pharmaceutical formulations of photosensitizers for systemic administration. Since most existing photosensitizers are poorly water soluble, they aggregate easily under physiological conditions and thus cannot be simply injected intravenously. Moreover, even with water-soluble photosensitizers, the accumulation selectivity for diseased tissues is not high enough for clinical use.

Photodynamic therapy (PDT) is based on the concept that certain therapeutic molecules called photosensitizers (photosensitizer) can be preferentially localized in malignant tissues, and when these photosensitizers are activated with appropriate wavelength of light, they pass on their excess energy to surrounding molecular oxygen resulting in the generation of reactive oxygen species (ROS), such as free radicals and singlet oxygen (¹O₂), which are toxic to cells and tissues.

PDT is a non-invasive treatment and used for several types of cancers, and its advantage lies in the inherent dual selectivity. First, selectivity is achieved by a preferential localization of the photosensitizer in target tissue (e.g. cancer), and second, the photoirradiation and subsequent photodynamic action can be limited to a specific area. Since the photosensitizer is non-toxic without light exposure, only the irradiated areas will be affected, even if the photosensitizer does infiltrate normal tissues.

Although PDT is emerging as a choice of treatment for many cancer patients, because of the hydrophobic nature of the most of the photosensitizers, searches are still on for developing an ideal photosensitizer formulation that can be easily injectable in vivo. Numerous approaches have been proposed to achieve not only stable aqueous dispersion but also site-specific and time-controlled delivery of therapeutic agents, often using a biocompatible delivery vehicle. Colloidal carriers for photosensitizers, such as oil-dispersions, liposomes, low-density lipoproteins, polymeric micelles, and recently ceramic nanoparticles are examples of delivery shuttles for photosensitizer molecules some of which may offer benefits from rendering aqueous stability and appropriate size for passive targeting to tumor tissues by the “enhanced permeability and retention” (EPR) effect, offering a possibility of bioconjugation approaches to enhance bioavailability as well as tumor targeting and offering a possibility of actively targeting tumor tissues by appropriate surface functionalization.

In PDT the release of the photosensitizer drugs is not a prerequisite for their therapeutic action (unlike in conventional chemotherapy), and the premature release of the photosensitizer molecules from carrier vehicles while in systemic circulation results in reduced efficacy of treatment.

Nanoparticles made of an organically modified silica complexed with polynucleotides have been described in co-pending U.S. priority application Ser. No. 11/195,066. That patent application does not, however, suggest anything concerning nanoparticles made of an organically modified silica with a photodynamic agent.

The results presented enable new and powerful treatment modalities for human neoplastic disease through development of novel multifunctional nanoparticles that are custom-tailored to target tumor cells and transport the therapeutic (including PDT agents, chemotherapeutic agents etc.) and/or imaging moieties for a “See and Treat” approach. The multifunctional device containing the PET/SPECT and or fluorescence capabilities will enable real-time imaging and monitoring the tumors before and after the treatment. To date, relatively little work has been done on the development of nanoparticles with such combined functionality.

Both cancer detection and treatment depend on the selective delivery of appropriate agents to the malignancy. Photodynamic therapy (PDT), a relatively new modality for the treatment of variety of oncological, cardiovascular, dermatological and ophthalmic diseases, is based on the preferential localization of the photosensitizing molecules in target tissue. We and others have developed relatively tumor-avid photosensitizers which selectively accumulate in tumors in vivo, and these molecules have been used to carry optical, PET and MR imaging agents to the tumor sites. However, the tumor selectivity of the current photosensitizers is not always adequate. Approaches of linking photosensitizer to antibody fragments or receptor ligands have been disappointing because the number of required photosensitizer/cell generally is greater than the number of antigen or receptor binding sites. Conversely, the imaging agent carrying capacity of individual photosensitizer molecules is limited.

Nanotechnology platforms potentially can deliver large number of photosensitizer and or/imaging agents. Nanoparticles are uniquely promising in that (i) their hydrophilicility and charge can be altered; (ii) they possess enormous surface areas and their surface can be modified with functional groups possessing a diverse array of chemical and biochemical properties including tumor selective ligands. There the aims of this invention was to develop biocompatible organically modified silicate sol (ORMOSIL) as multifunctional nanovectors for tumor therapy and imaging (optical, PET, SPECT and MR). The tumor therapy is not limited to PDT and it could also be chemotherapy, radiotherapy, depending upon the characteristics of the post-loaded or conjugated nanoparticles.

The integration of nanoscience and nanotechnology in biomedical research is ushering in a true revolution that is broadly impacting biotechnology. New terms such as nanobioscience, nanobiotechnology and nanomedicine have recently come into existence and are rapidly gaining wide acceptance. Nanochemistry deals with the confinement of chemical reactions to produce nanometer-scale products (generally 1-100 nm size range). The challenge is to be able to use these nanochemical approaches to reproducibly provide precise control of composition, size, and shape of the nano-objects formed.

In the field of nanotechnology, silica provides a number of advantages as the shell material in the fabrication of nanoparticles. A key advantage is the ease of synthesis that requires no special reaction conditions such as inert atmosphere, high temperature etc. As a result, properties like particle size and surface termination can be very easily manipulated using different silica precursors (tetramethyl orthosilicates, tetraethyl orthosilicates etc.), the hydrolysis reaction can be easily controlled, and it is the tenability of these parameters that makes an ideal candidate for use as a shell in nanoparticle synthesis. The peripheral surface as well as the inner core can be made hydrophilic or hydrophobic as per nature of the therapeutic or imaging candidate(s).

### Cancer Therapy:

The major challenge of cancer therapy is preferential destruction of malignant cells with sparing the normal tissue. Critical for successful eradication of malignant disease are early detection and selective ablation of malignancy. PDT is a clinically effective, and still evolving, locally selective therapy of cancers. It is FDA approved for early and late stage lung cancer, obstructive esophageal cancer, high-grade dysplasia associated with Barrett's esophagus, age-related macular degeneration and actinic keratoses. PDT employs tumor localizing photosensitizers that produce reactive singlet oxygen upon absorption of light. Subsequent oxidation-reduction reactions also can produce superoxide anions, hydrogen peroxide and hydroxyl radicals. The preferential killing of the target cells (e.g. tumor), rather than adjacent normal tissues, is essential for PDT, and the preferential target damage achieved in clinical applications is a major driving force behind the use of this modality. The success of PDT relies on development of tumor-avid molecules that are preferentially retained in malignant cells but cleared from normal tissues.

Clinical PDT initially was developed at RPCI, and we have one of the world's largest basic and clinical research program. Initially the RPCI group developed Photofrin®, the first generation FDA approved hematoporphyrin-based compound. Subsequently, our group has investigated the structure activity relationships for tumor selectivity and photosensitizing efficacy, and used the information to design new photosensitizers with high selectivity and desirable pharmacokinetics. Although the mechanism of porphyrin retention by tumors in not well understood, the balance between lipophilicity and hydrophilicity is recognized as an important factor. In our efforts to develop effective photosensitizers with required photophysical characteristics, we used chlorophyll-a and bacteriochlorophyll-a as the substrates. An extensive QSAR studies on a series of the alkyl ether derivatives of pyropheophorbide-a (660 nm) led to selection of the best candidate, HPPH (hexyl ether derivative) currently in promising Phase II clinical trials. Our PS development currently is being extended in purpurinimide (700 nm) and bacteriopurpurinimde (780-800 nm) series with high singlet oxygen (¹O₂) producing capability. The long wavelength absorption is important for treating large deep-seated tumors, because it both increases light penetration and minimizes the number of optical fibers needed for light delivery within the tumor. Some of these compounds are highly tumor avid. We have previously shown that at 48 and 72 h after administration we are able to achieve ratios ˜6:1 and 10:1 between the tumor and surrounding muscle.

### Cancer Imaging

### Optical Imaging.

Optical imaging using bioluminescent or fluorescent probes is a rapidly evolving field, particularly for small animals. Fluorescent probes offer the advantage of near infrared wavelengths, where light penetration into and out of tissue is very high. For small animals, planar images are adequate, but optical tomographic reconstructions of fluorescent images is becoming feasible.

PS generally fluoresce and the fluorescence properties of these porphyrins in vivo has been exploited by several investigators for the detection of early-stage cancers in the lung, bladder and various other sites. In addition, for treatment of early disease or for deep seated tumors the fluorescence can be used to guide the activating light. However, photosensitizers are not optimal fluorophores for tumor detection for several reasons: (1) They have low quantum yields. Because the excited state energy is transferred to the triplet state and then to molecular oxygen, efficient photosensitizers tend to have lower fluorescence efficiency (quantum yield) than compounds designed to be fluorophores, such as cyanine dyes. (2) they have small Stokes shifts. Phorphyrin-based photosensitizers have a relatively small difference between the long wavelength absorption band and the fluorescence wavelength (Stokes shift), which makes it technically difficult to separate the fluorescence from the excitation wavelength. (3) They have relatively short fluorescent wavelengths, <800 nm, which are not optimal for deep tissue penetration. Thus, for effective optical imaging NP with or without PS require additional fluorophores. For in vivo use we seek fluorophores with both excitation and emission>600 nm; for deep tissue light penetration the wavelengths should be in the near infrared (NIR), >750 nm. Although quantum dots can emit in the NIR, they are best excited with short wavelengths, their toxicity is problematic and their incorporation within small NP may be difficult. Thus we plan to utilize high extinction coefficient NIR cyanine dyes. In the NP formulations these dyes will permit tracking the particles in vivo and ex vivo in tissue sections, as well as making it possible to optically detect malignancies.

### Molecular Imaging Using Animal PET:

Positron emission tomography (PET) is a technique that permits non-invasive use of radioisotope labeled molecular imaging probes to image and assay biochemical processes at the level of cellular function in living subjects. Currently, PET is important in clinical care and is a critical component biomedical research, supporting a wide range of applications, including studies of gene expression, perfusion, metabolism and substrate utilization, neurotransmitters, neural activation and plasticity, receptors and antibodies, stem cell trafficking, tumor hypoxia, apoptosis and angiogenesis. PET predominately has been used as a metabolic marker, without specific targeting to malignancies. In part this is because of the short half lives of most of the isotopes used for imaging. However, we have successfully employed ¹²⁴I, with a half-life of 4.2 days, devised a coupling reaction which rapidly and efficiently links it to a tumor-avid PS, and used the conjugate to target and image tumors. The approach should be directly applicable to targeted NP formulations, where the much higher isotope payload will improve the imaging sensitivity.

### Ormosil NPs:

Organically modified silica (Ormosil) is synthesized from precursor organosilane molecules where one or two of the alkoxy groups of a tetra-alkoxysilane molecule have been replaced by hydrocarbon groups. ORMOSIL nanoparticles have the potential to overcome many limitations of their ‘un-modified’ silica counterparts. The presence of both hydrophobic and hydrophilic groups on the precursor alkoxy-organosilane helps them to self-assemble both as normal micelles and reverse micelles under appropriate conditions. The resulting micellar (and reverse micellar) cores can be loaded with biomolecules like drugs, proteins, etc. Such a system has a number of advantages: (a) they can be loaded with either hydrophilic or hydrophobic drugs/dyes; (b) they can be precipitated in oil-in-water microemulsions where corrosive solvents like cyclohexane and complex purification steps like solvent evaporation, ultra-centrifugation etc., can be avoided; (c) their organic groups can be further modified for attachment of targeting molecules; and (d) they can be possibly bio-degraded through the biochemical decomposition of the Si—C bond (13). The presence of the organic group also imparts some degree of flexibility to the otherwise rigid silica matrix, which is expected to enhance the stability of such particles in aqueous systems against precipitation.

### (i) Preparation of Ormosil NP by Normal Micellar Methods.

ORMOSIL NP were prepared in the non-polar aqueous core of oil-in water microemulsions using the well established tween 80/butanol/water system. For ORMOSIL nanoparticles having —OH group on the surface, hydrophobic dye or PS is added in DMSO, and the NP were precipitated by adding 30 ul of ammonia. On the other hand amino-terminated ORMOSIL nanoparticles were precipitated by adding a calculated amount of 3-aminopropyltriethoxysilane and stirring for about 20 hours at room temperature (FIG. 1). After the formation of the nanoparticles, surfactant tween 80 and co-surfactant 1-butanol were removed by dialyzing the solution against water in a 12-14 kDa cutoff cellulose membrane (Spectrum Laboratories, Inc.) for 50 h. The dialyzed solution was then filtered through a 0.2 μm cut-off membrane filter (Nalgene) and used straightaway for further experimentation. The hydrophobic fluorescent dyes remain encapsulated within the ORMOSIL matrix, rendering the NP fluorescent.

iii) Preparation Of Ormosil Nanoparticles by Reverse Micellar Methods.

For the encapsulation of hydrophilic dye/and have the vinyl groups on the surface for further functionalization, ORMOSIL nanoparticles were prepared in the aqueous core of the reverse micellar droplets (Sharma et al 2004). (FIG. 2). In a typical experiment, 20 ml of 2% aqueous Tween 80, 400 ul of the dye solution in water (or only the DI water in case of void nanoparticles) and 500 ul VTES and 200 ul of ammonia were added. The whole solution was stirred for 72 hours for the completion of the reaction. The silica nanoparticles were separated by centrifugation and repeatedly washed with hexane to remove the surfactant and unreacted materials. These Ormosil NP thus formed are not only capable of encapsulating hydrophilic fluorescent molecules, but also make the vinyl groups available on the surface for further functionalization.

### (i) Photosensitizer Encapsulated Nanoparticles:

We have previously reported a highly stable aqueous formulation of organically modified silica (ORMOSIL) nanoparticles encapsulating the hydrophobic PS HPPH [2-devinyl-2-(1-′-hexyloxyethyl)pyropheophorbide, where the encapsulated photosensitizers is able to generate singlet oxygen upon photoactivation owing to the free diffusion of molecular oxygen across the ORMOSIL matrix. However because of the mesoporosity of the ORMOSIL matrix, encapsulation of the PS does not exclude the PS release, at least partially, during systemic circulation (FIG. 3).

### BRIEF DESCRIPTION OF THE INVENTION

In accordance with the invention, nanoparticles postloaded with photosensitizer molecules are provided to overcome the drawback of their premature release and thus enhance the outcome of PDT.

In accordance with the invention, silica sol-gel based nanoparticles are provided containing at least one post-loaded photosensitizer. The photosensitizer is preferably a tetrapyrrole-based compound related to porphyrins, chlorins, bacteriochlorins, benzochlorins, benzoporphyrin derivatives, pheophorbides including pyropheophorbides, and phthalocyanines, naphthanocyanines with and without fused ring systems and derivatives of all the above.

The nanoparticle may also include imaging agents, e.g. radionuclides, magnetic resonance (MR) and fluorescence imaging agents, either post-loaded or chemically bonded. The imaging agents and photosensitizers may be at a periphery (surface) of the nanoparticles to increase efficiency.

Target-specific nanoparticles may be provided by incorporating biotargeting molecules such as specific antibodies at the surface that react with particular ligands to obtain target specificity. Diagnostic agents may be present in the antibody in addition to imaging agents and tumor specific photosensitizers as previously and subsequently discussed.

In general, the nanoparticle of the invention has the structural formula:

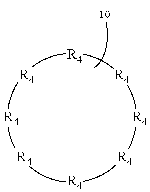

where the ring represents a siloxane polymer matrix.

R₄ is (R₁)ₙ-(R₂)ₙ where R₁ may be a labeled photosensitizer (IP) or unlabeled photosensitizer (P), cyanine dye, SPECT (single proton emission computed tomography) imaging agent, PET (positron emission tomography) imaging agent, MR imaging agent or fluorescent imaging agent at least partially available at a surface of the siloxane polymer matrix.

At least one R₁ or R₂ group is a photosensitizer, preferably a tetrapyrollic photosensitizer, e.g. porphyrins, chlorins, bacteriochlorins, benzochlorins, benzoporphyrins, pheophorbides including pyropheophorbides, and derivatives thereof.

n is 0 or 1; provided that at least one n is 1;

R₂ is cyanine dye, SPECT, PET, MR or fluorescent imaging agent, linked targeting agent RGD, F3 peptide, carbohydrate or folic acid or labeled photosensitizer (IP) or unlabeled photosensitizer (P) post loaded so as to be at least partially embedded in the siloxane polymer matrix. RGD is a peptide that contains the Arg-Gly-Asp attachment site that recognizes v3 and v5 integrin receptors that play a role in angiogenesis, vascular intima thickening and proliferation of malignant tumors.

At least one labeled photosensitizer (IP) or unlabeled photosensitizer (P) is present in R₁ or R₂ that is sufficiently embedded in the siloxane polymer matrix by postloading to prevent leaching to an extent greater than 40% upon 24 hour continuous washing in 1% bovine serum albumin (BSA).

### BRIEF DESCRIPTION OF THE DRAWINGS

FIG. 1 a schematic diagram for the preparation of Ormosil (silane) nanoparticles by a normal micellar method. a) represents dye in AOT/BuOH/water micelles. b) represents nanoparticles in aqueous dispersion. c) represents amino terminated nanoparticles in aqueous dispersion

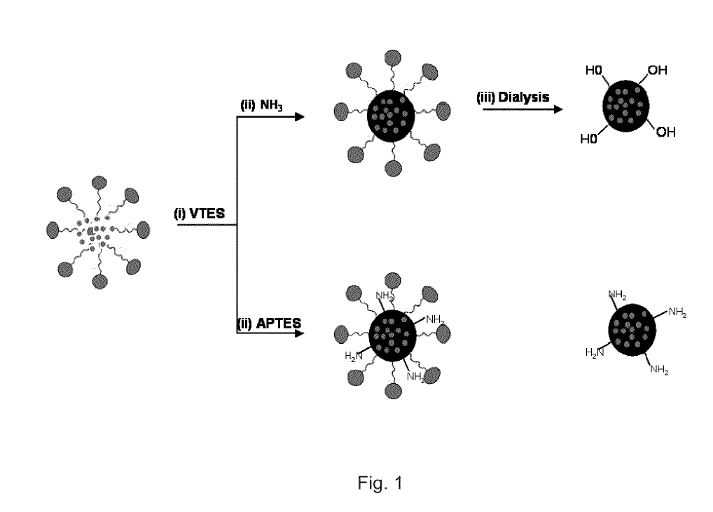

FIG. 2 shows a schematic diagram for preparation of Ormosil nanoparticles by a reverse micellar method.

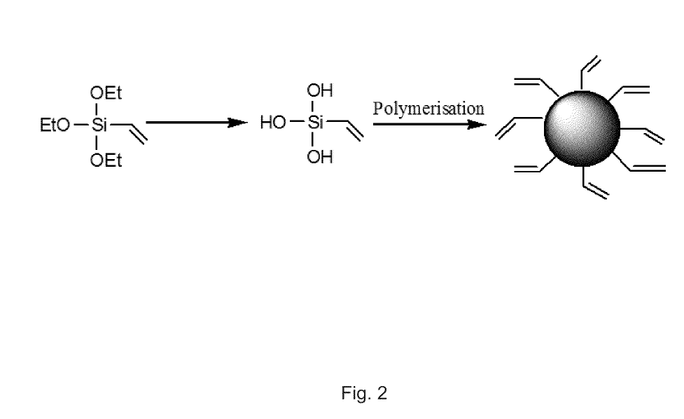

FIG. 3 shows photosensitizers encapsulated in Ormosil nanoparticles. a) represents nanoparticles. b) represents photosensitizer. c) represents ORMOSIL Nanoparticles.

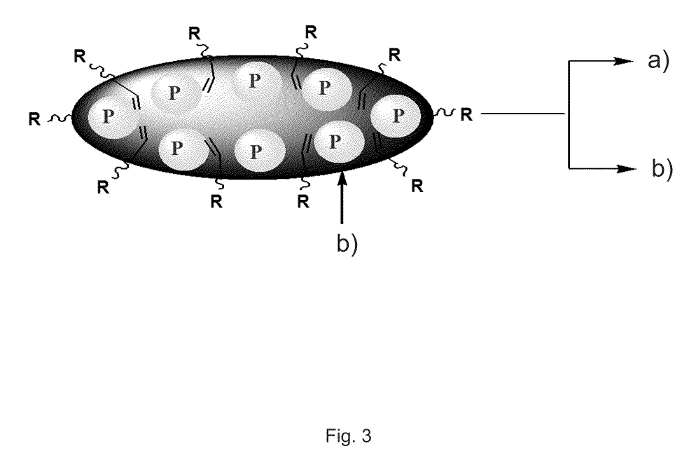

FIG. 4 shows structures of examples of photosensitizers and cyanine dye that can be postloaded into Ormosil nanoparticles.

FIG. 5 shows examples of photosensitizer encapsulated Omosil nanoparticles that may have tumor-target specificity. R represents —COOH, —OH, CH═CH₂, —NH₂, —SH or tumor targeting moiety. a) represents an ORMOSIL nanoparticle.

FIG. 6 shows examples of cyanine dye post-loaded Ormosil nanoparticles that may have tumor-target specificity. R represents —COOH, —OH, CH═CH₂, —NH₂, —SH or tumor targeting moiety. a) represents cyanine dye and b) represents an ORMOSIL nanoparticle.

FIG. 7 shows examples of photosensitizer and cyanine dye post loaded into ORMOSIL nanoparticles as bifunctional agents that may also have tumor-target specificity. R represents —COOH, —OH, CH═CH₂, —NH₂, —SH or tumor targeting moiety. a) represents cyanine dye and b) represents an ORMOSIL nanoparticle.

FIG. 8 shows photosensitizer conjugated (with and without ¹²⁴I) and the cyanine dye (with and without tumor targeting moieties) in post-loaded ORMOSIL nanoparticles as multivalent agents (PET, fluorescence, PDT and target-specificity). R represents —COOH, —OH, CH═CH₂, —NH₂, —SH or tumor targeting moiety. a) represents cyanine dye and b) represents an ORMOSIL nanoparticle.

FIG. 9a shows a TEM image of purpurinimide 1 postloaded in Omosil nanoparticles, size 20-25 nm.

FIG. 9b shows a TEM image of bacteriopurpurinimide 2 postloaded in Omosil nanoparticles, size 20-25 nm.

FIG. 10 shows graphs of release kinetics for bacteriopurpurinimide 2 at graph A and purpurinimide 1 at graph B. Where Y represents % intensity at 785 nm in A and 700 nm in B and X=time 9 of incubation

FIG. 11 shows release kinetics of cyanine dye 9 post-loaded in Ormosil nanoparticles. Y represents % intensity at 797 nm. In A a) represents control, b) represents miscelles and c) represents NP's. In D a) represents control, b) represents dye released and c) represents dye in NP's. Y represents % intensity at 797 nm. X=time of reaction. a) is a control, b) is free dye and c) is loaded dye.

FIG. 12 is a graph showing release of post-loaded cyanine dye X from the HPPH-conjugated ORMOSIL nanoparticles, where a) is a control b) is free dye and c) is loaded dye.

FIG. 13 shows absorption spectrum of nanoparticles containing both HPPH (415 and 660 nm) and the cyanine dye [730-820 nm (broad)]. X=intensity. Y=wavelength in nm.

FIG. 14 is a graph showing that no decay of the cyanine dye, during the post-loading in ORMOSIL nanoparticles, was observed. X=intensity at 797 nm. Y=time of reaction

FIG. 15 shows (A) fluorescence spectra of HPPH in 1% Tween 80 (control), HPPH-postloaded and HPPH-cationic post loaded nanoparticles at equal concentration and (B) post-loading efficiency of HPPH 7 cationic-HPPH 8. In A X=wavelength in nm and Y=fluorescence intensity in nm. In B, Y═OD at 663 nm. a)=control. b)=miscelles and c)=NP's

FIG. 16 shows comparative release of encapsulated and post-loaded photosensitizers in ORMOSIL nanoparticles. Bar graph series 1 is HPPH encapsulated. Bar graph series 2 is HPPH post loaded and bar graph series 3 is HPPH cationic post loaded. a)=control. b)=wash 1. c)=wash 2. d)=wash 3. e)=wash 4. f)=wash 5. g)=wash 6 and h)=retention.

FIG. 17 shows in vitro photosensitizing efficacy of HPPH, LG-114 (NPs without any photosensitizer, LG 115 E-=ORM (HPPH-encapsulated NPs), LG-116 PL-ORM (HPPH-post-loaded NPs). The photodynamic therapy (PDT) was of colon-26 cells (1 μM HPPH PS/NP), 4 h incubation, n=3, mean+/−SEM). X=light dose (J/cm²). Y=fraction surviving.

FIG. 18 shows in vitro photosensitizing efficacy of purpurinimide 1 and bacteriopurpurinimide 2 in Colon-26 cells. X=light dose (J/cm²). Y=percent surviving. a)=control,

FIG. 19 shows In vivo photosensitizing efficacy of purpurinimide 1 and bacteriopurpurinimide 2 in BALB/c mice bearing RIF tumors. X=days after treatment and Y=% mice without tumor regrowth. a)=control.

FIG. 20 shows a fluorescence image of Colon-26 tumor implanted in BALB-c mice with HPPH-post-loaded ORMOSIL NPs at 24 h post injection (drug dose: 0.47 μmol/kg)

FIG. 21 shows photosensitizer post-loaded ORMOSIL nanoparticles with tumor-targeted specificity. R represents —COOH, —OH, CH═CH₂, —NH₂, —SH or tumor targeting moiety. b) represents an ORMOSIL nanoparticle.

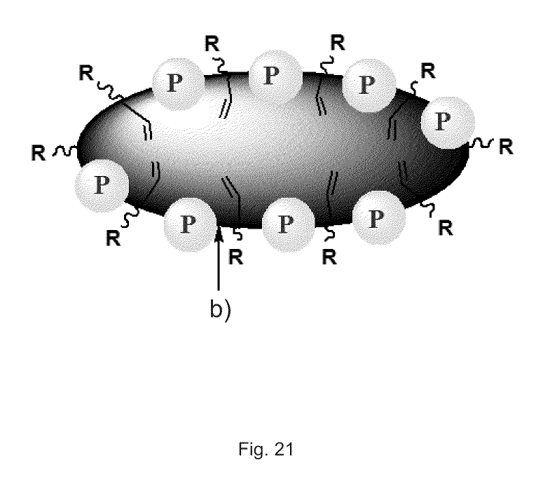

FIG. 22 shows cyanine dye post-loaded ORMOSIL nanoparticles with tumor-target specificity. R represents —COOH, —OH, CH═CH₂, —NH₂, —SH or tumor targeting moiety. a) represents cyanine dye and b) represents an ORMOSIL nanoparticle.

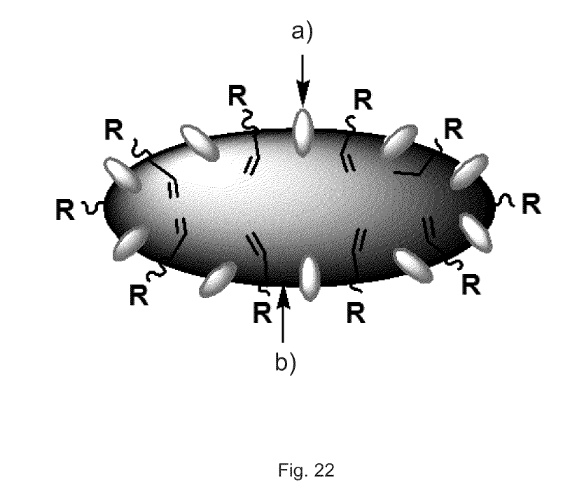

FIG. 23 shows photosensitizer and cyanine dye post loaded ORMOSIL nanoparticles as bifunctional agents with tumor-target specificity R represents —COOH, —OH, CH═CH₂, —NH₂, —SH or tumor targeting moiety. a) represents cyanine dye and b) represents an ORMOSIL nanoparticle.

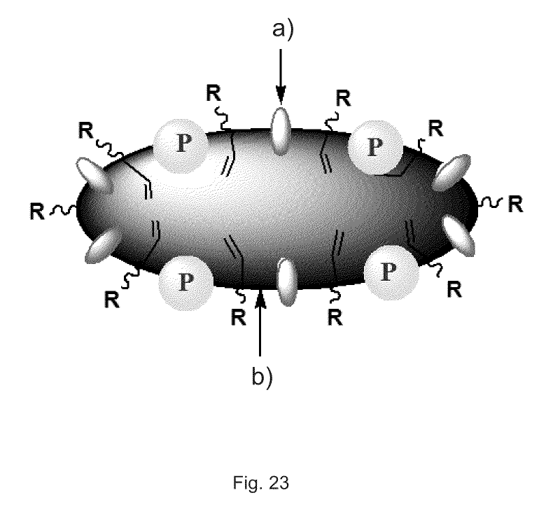

FIG. 24 shows photosensitizer conjugated (with and without ¹²⁴I) and the cyanine dye post-loaded ORMOSIL nanoparticles as multivalent agents (PET, fluorescence, PDT and target-specificity). R represents —COOH, —OH, CH═CH₂, —NH₂, —SH or tumor targeting moiety. a) represents cyanine dye and b) represents an ORMOSIL nanoparticle.

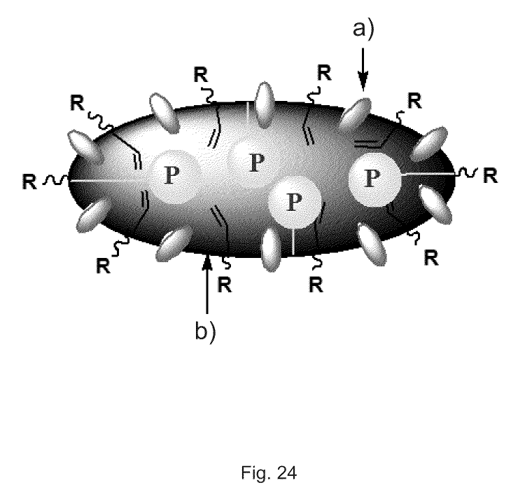

FIG. 25 shows schematic structures of examples of photosensitizers that can be post loaded into silane based nanoparticles. In compounds a), b), and structures c) R₁=an alkyl chain with variable saturated or unsaturated carbon units, —CH(OR₃)CH₃, where R₃ is alkyl with variable carbon units 1-12. and aryl, heterocyclic ring systems and/or a substituted aryl group I nd I-124 substituents. R₂═—OH, —OMe, or —NHR₄ where R₄=-folic acid, cyclic or linear amino acid including RGD and F3 peptides. R₅=alkyl, aryl, substituted aryl, linear or cyclic amino acid, heteraromatic ring systems, —COOH, CO₂Me, or —CONHR₇ where R₇=various amino acids. M=2H or various metals including Ga, Al, Ni, Cu, Sn, Zn, etc., E=open chain and fused isocyclic and N-substituted imide ring systems. R₆═R₅ or CH₂COOH, or —CH₂CO₂Me. In compounds d) and e) showing phthalocyanines and naphthalocyanines, R=various hydrophilic and hydrophobic groups including iodinated substituents with and without 1-124 labels. M=various metals and substituted metals linked with tumor targeting groups including peptides and folic acids, etc. In compounds f) and g) showing cyanine dyes, R═—COOH, —OH, —NH₂, —SH, Cl, or substituted or unsubstituted aromatic or photosensitizer. R₁═—COOH, —OH, —NH₂, —SH, Cl, or —SO₃H. X=various aromatic and heteroaromatic moieties.

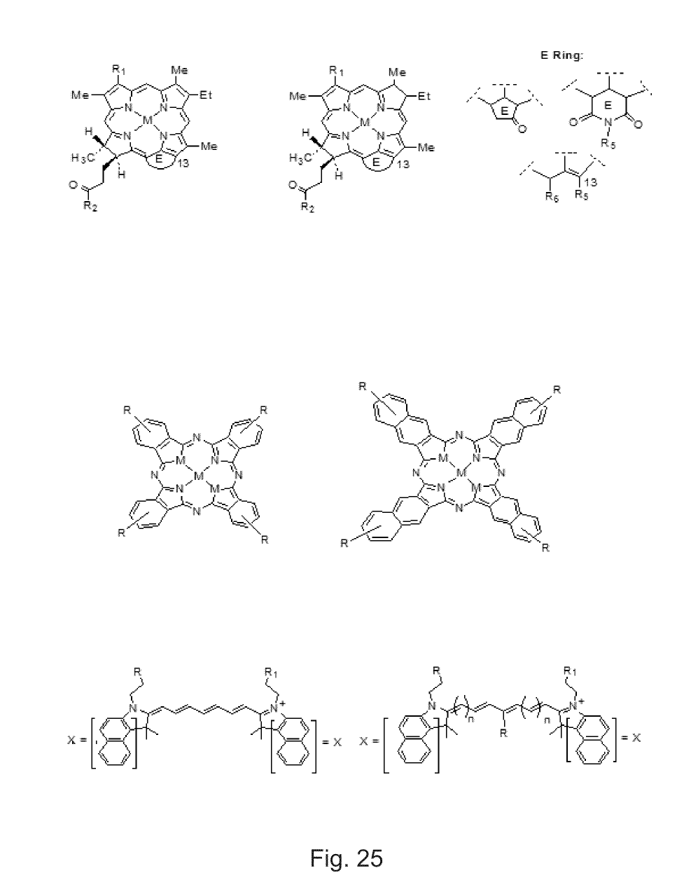

### DETAILED DESCRIPTION OF THE INVENTION

As previously discussed, in accordance with the invention, silica-based nanoparticles are provided containing at least one post loaded photosensitizer. The photosensitizer is usually a tetrapyrrole-based compound, a phthalocyanines or naphthanocyanines with and without fused ring systems and derivatives of all the above. The photosensitizer is preferably related to porphyrins, chlorins, bacteriochlorins, benzochlorins, benzoporphyrin derivatives, pheophorbides including pyropheophorbides. Specific examples of such tetrapyrollic photosensitizers may be found in numerous U.S. patents, e.g. U.S. Pat. Nos. 5,864,035; 5,952,366; 6,533,040; 6,624,187; and RE39,094;

The nanoparticle may also include covalently linked imaging agents, e.g. radionuclides, magnetic resonance (MR) and fluorescence imaging agents. The imaging agents and photosensitizers may be at a periphery (surface) of the nanoparticles to increase efficiency.

Target-specific nanoparticles may be provided by incorporating biotargeting molecules such as specific antibodies at the surface that react with particular ligands to obtain target specificity. Diagnostic agents may be present in the antibody in addition to imaging agents and tumor specific photosensitizers as previously and subsequently discussed.

In general, the nanoparticle of the invention has the structural formula:

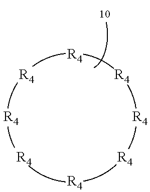

where the ring represents a siloxane matrix that may be considered a sol gel.

R₄ is (R₁)ₙ-(R₂)ₙ where R₁ is a labeled photosensitizer (IP) or unlabeled photosensitizer (P), cyanine dye, SPECT (single proton emission computed tomography) imaging agent, PET (positron emission tomography) imaging agent, MR imaging agent or fluorescent imaging agent at least partially available at a surface of the siloxane polymer matrix.

At least one R₁ group may be a tetrapyrollic photosensitizer, e.g. porphyrins, chlorins, bacteriochlorins, benzochlorins, benzoporphyrins, pheophorbides including pyropheophorbides, and derivatives thereof.

n is 0 or 1; provided that, at least one n is 1 and the compound contains at least one labeled or unlabeled photosensitizer.

R₂ is cyanine dye, SPECT, PET, MR or fluorescent imaging agent, linked targeting agent RGD, F3 peptide, carbohydrate or folic acid or labeled photosensitizer (IP) or unlabeled photosensitizer (P) post loaded so as to be at least partially embedded in the siloxane polymer matrix. RGD is a peptide that contains the Arg-Gly-Asp attachment site that recognizes v3 and v5 integrin receptors that play a role in angiogenesis, vascular intima thickening and proliferation of malignant tumors.

The R₁ or R₂ group may be phthalocyanine, naphthanocyanine and derivatives thereof and may also be a radionuclide or MR or fluorescencent imaging agent. A plurality of R₁ groups are preferably photosensitizers located at peripheral positions on the nanoparticle and a plurality of R₂ groups are imaging agents located at peripheral positions on the nanoparticle.

The nanoparticle are desirably provided with biotargeting molecules following suitable surface functionalization to obtain target-specific nanoparticles. Examples of such biotargeting molecules are antibodys and the suitable surface functionalization for the antibody is a ligand, e.g. RGD and F3 peptide.

The nanoparticle may further include at least one diagnostic agent.

“Photosensitiers” (PS) as used herein means any material that can enter or attach to a cell or portion thereof and be activated by electromagnetic radiation, usually light, to destroy the cell or significantly alter its activity.

“nanoparticles made of an organically modified silica” as used herein refers to nanoparticles made from silica that has been organically modified to self organize into polysiloxane nanoparticles upon precipitation from solution. Preferred organically modified silica nanoparticles are ORMOSIL nanoparticles usually made by inclusion of a vinyltriethoxysilane in a surfactant solution followed by precipitation with ammonia or other amine, e.g. 3 aminopropyltriethoxysilane. In the first case the nanoparticle has surface —OH groups and in the second case has surface amino groups.

“SPECT” means “single proton emission computed tomography” imaging agent.

“PET” means “positron emission tomography” imaging agent.

“MR” means “magnetic resonance” imaging agent.

“RGD” refers to a peptide that contains the Arg-Gly-Asp attachment site that recognizes v3 and v5 integrin receptors that play a role in angiogenesis, vascular intima thickening and proliferation of malignant tumors.

The polysiloxane matrix is formed by self reaction of oxysilanes by dehydration (condensation) to form a polysiloxane matrix of silicon atoms interconnected by oxygen atoms. The starting oxysilanes have the formula R₄Si where R is independently at each occurrence an alkyl, alkylene, hydroxy or alkoxy group, provided that at least two of said R groups are hydroxy groups. The other R groups are usually hydroxy, alkoxy or an alkyl group substituted with an alkoxy, carboxy, hydroxyl, amino or mercapto group. The silanes and R groups are selected such that they will form nanoparticles having a size of less than 200 nm, preferably less than 100 nm and most preferably less than 50 nm. Particles of a size less than 20 nm are most desirable in most circumstances. The silanes, usually oxysilanes, are selected so that the nanoparticles will have hydroxyl, amino, mercapto and/or carboxy groups exposed at its surface.

The oxysilane is desirably selected from the group consisting of vinyltrimethoxysilane, vinyltriethoxysilane, γ-glycidoxypropyltrimethoxysilane, γ-methacryloxypropyltrimethoxysilane, γ-aminopropyltrimethoxysilane, γ-aminopropyltriethoxysilane, γ-mercaptopropyl-trimethoxysilane, γ-3,4-epoxycyclohexyltrimethoxysilane and phenyltrimethoxysilane.

A general approach for post loading the photosensitizers and imaging agents in nanoparticles with and without targeting functionality is as follows:

ORMOSIL precursor vinyltriethoxysilane (VTES) and other reagents were purchased from Sigma-Aldrich and were used without any further purification. Microfuge membrane-filters (NANOSEP 100K OMEGA) are a product of Pall Corporation.

In general, the nanoparticles were synthesized by the alkaline hydrolysis and polycondensation of the organo-trialkoxysilane precursors within the non-polar core of Tween-80/water microemulsion, with the protocol similar to that reported previously. Briefly, a mixture 10 ml of 2% aqueous Tween-80 solution, 300 mL of co-surfactant 1-butanol were vigorous stirred. To this solution, 100 mL of DMSO was added. After 10 min, 100 μl of vinyltriethoxysilane (VTES) was added dropwise and the resulting mixture was magnetically stirred for one hour. At this stage, 20 mL of aqueous ammonia was added and the resulting solution was magnetically stirred overnight for the formation of the nanoparticles. Then, the nanoparticles were dialyzed for 24 hrs against distilled water using a cellulose membrane of cut-off pore size of 12-14 kD for the removal of unreacted starting materials. The dialysate containing the ORMOSIL nanoparticles was sterile filtered (0.2 μM membrane) and was stored at 4° C. for further use. 5 ml of above solution of blank nanoparticles was taken in a vial and 50 μL of 10 mmol DMSO solution of PS was added and resultant mixture was stirred for 12 hrs. Then, the post loaded nanoparticles were dialyzed for 24 hrs against distilled water using a cellulose membrane of cut-off pore size of 12-14 kD for the removal of residual DMSO and any loosely bound PS.

To separate the Tween-80 micelles (and any loosely bound PS) from the nanoparticles, the dialyzed dispersions were filtered and residue was washed three times with water through a microfuge membrane-filter (NANOSEP 100K OMEGA, Pall Corporation, USA) by centrifuging at 9,000 rpm for 30 minutes (spin-filtration). Tween-80 micelles and loosely bound PS molecules flow-through this membrane and are collected in the lower tube (flow-fraction), while nanoparticles get embedded in the membrane and can be subsequently extracted by adding water and sonicating/vortexing (membrane-fraction).

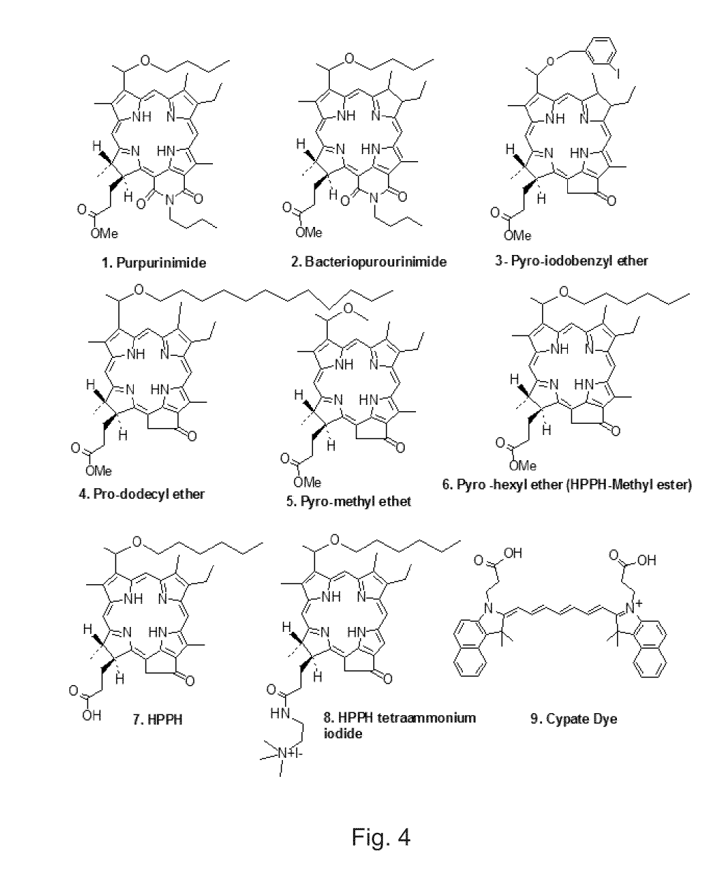

The photosensitizers shown in FIG. 4 were converted into a series of nanoparticles and the structures are illustrated with the following schematic representations:

### Category 1:

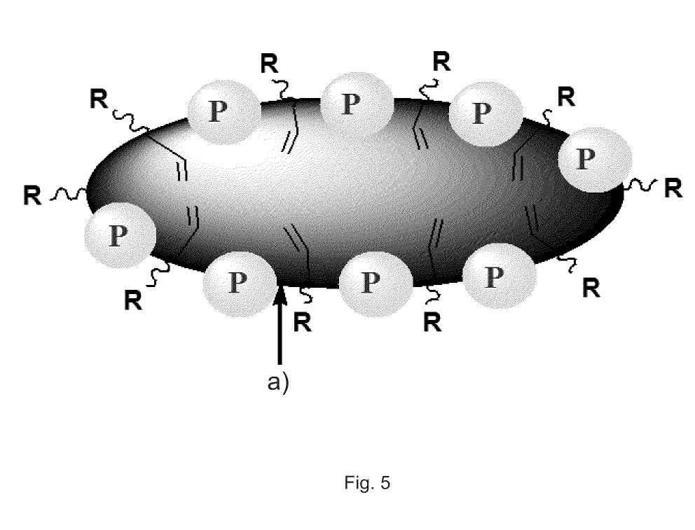

The functionalized ORMOSIL nanoparticles were prepared by the methodology discussed above and post loaded with photosensitizers with fluorescence imaging potential of superficial tumors. The main objective of this approach was to investigate the effect of targeting moieties (carbohydrates, peptides (RGD and F3 peptides) introduced at the peripheral position of the nanoparticles in tumor-specificity and photosensitizing efficacy (FIG. 5).

### Category 2:

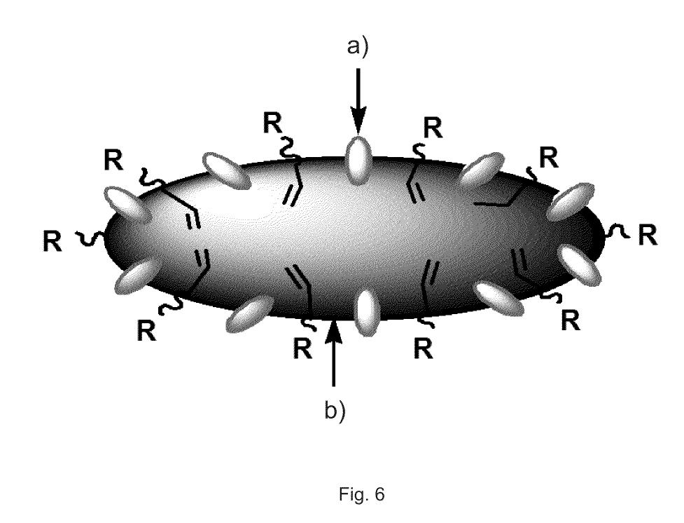

The functionalized ORMOSIL nanoparticles were prepared by the methodology discussed above and post loaded with a cyanine dye (e.g. cypate) for fluorescence imaging of peripheral and deeply seated tumors. The effect of target-specific moieties can be addressed by conjugating the target-specific agents at the peripheral position(s) of the nanoparticles (FIG. 6).

### Category 3:

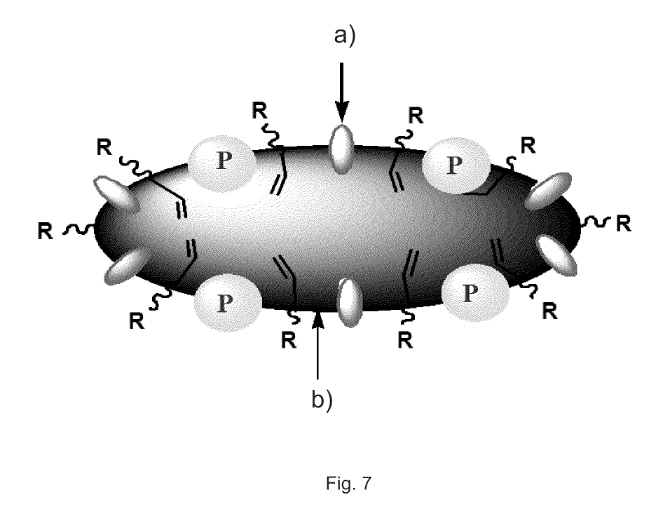

In this approach, ORMOSIL nanoparticles were post-loaded with the photosensitizers (with and without ¹²⁴I nuclide) and the cyanine dye. To investigate the impact of target-specific NPs, the targeting moieties were either introduced at the peripheral positions or the photosensitizers conjugated with targeting moieties were post loaded to ORMOSIl NPs (FIG. 7).

### Category 4:

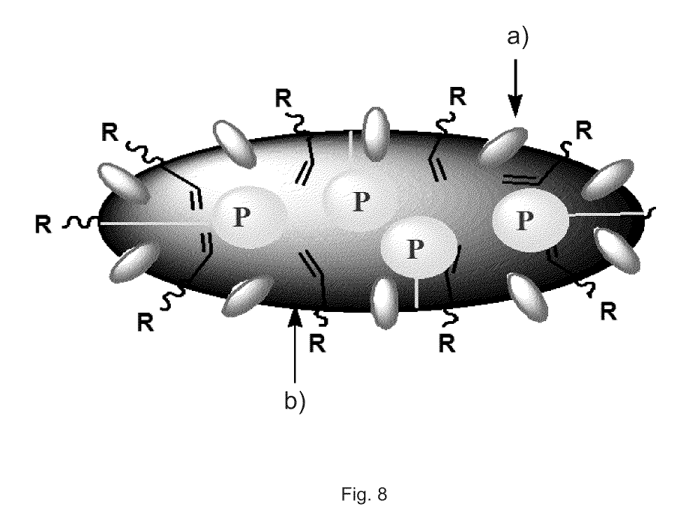

In this approach, the cyanine dyes (with and without tumor targeting moieties) were post-loaded to photosensitizers-conjugated nanoparticles with various functionalities (with and without tumor-targeting functionalities) at the peripheral position (FIG. 8).

### Characterization of Size, Shape and Functionality of the Nanoparticles.

Transmission electron microscopy (TEM) was performed to determine the size and shape of the prepared nanoparticles, using a JEOL JEM-100cx microscope at an accelerating voltage of 80 kV. UV-visible absorption spectra were acquired using a Shimadzu UV-3600 spectrophotometer, in a quartz cuvette with 1 cm path length. Fluorescence spectra were recorded on a Fluorolog-3 spectrofluorometer (Jobin Yvon, Longjumeau, France). Generation of singlet oxygen (¹O₂) was detected by its phosphorescence emission peaked at 1270 nm. A SPEX 270M Spectrometer (Jobin Yvon) equipped with a Hamamatsu IR-PMT was used for recording singlet oxygen phosphorescence. The sample solution in a quartz cuvette was placed directly in front of the entrance slit of the spectrometer and the emission signal was collected at 90-degrees relative to the exciting laser beam. An additional longpass filters (a 950LP filter and a 538AELP filter, both from Omega Optical) were used to attenuate the scattered light and fluorescence from the samples. ¹O₂ phosphorescence decays at 1270 nm were acquired using Infinium oscilloscope (Hewlett-Packard) coupled to the output of the PMT. A second harmonic (532 nm) from nanosecond pulsed Nd:YAG laser (Lotis TII, Belarus) operating at 20 Hz was used as the excitation source.

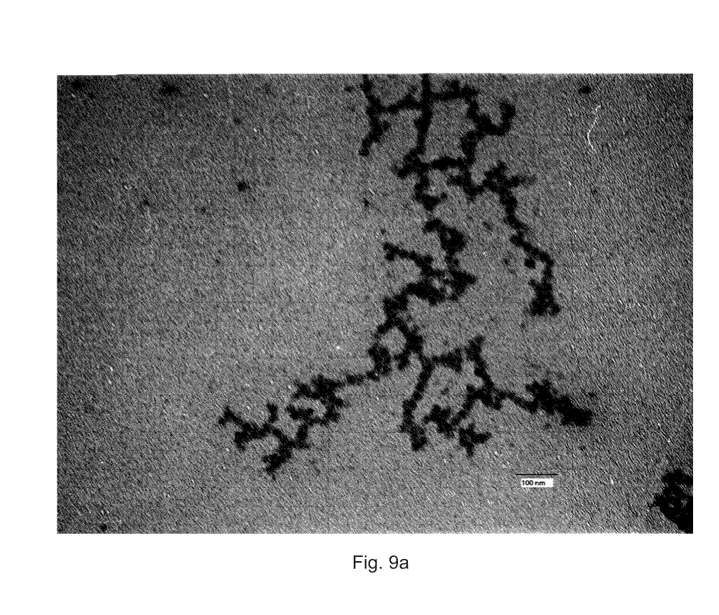

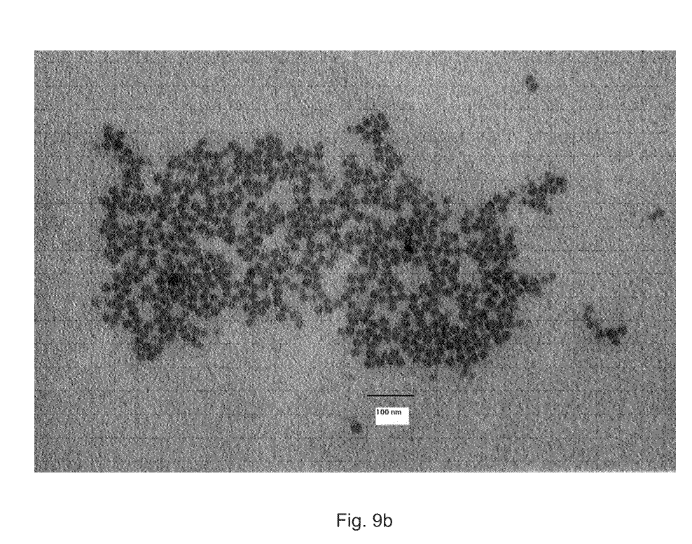

FIG. 9a show: a TEM images of purpurinimide 1 and FIG. 9b shows a TEM image of bacteriopurpurinimide 2, both post loaded ORMOSIL Nanoparticles. Size: 20-25 nm.

### Release Kinetics of Post-Loaded Nanoparticles:

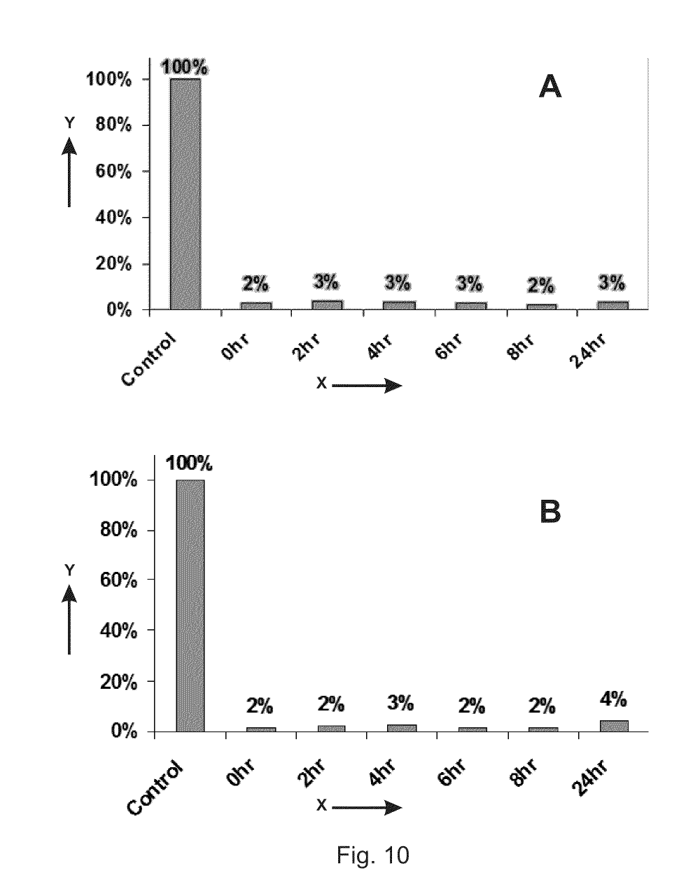

The release kinetics of some of the photosensitizers is shown in FIGS. 10-16.

Purpurinimide 1 and bacteriopurpurinimide 3: As can be seen from FIG. 10, both post-loaded photosensitizers retained in the ORMOSIL nanoparticles with a high concentration. Even on continuous washing, only a small percentage of the photosensitizers were leached out.

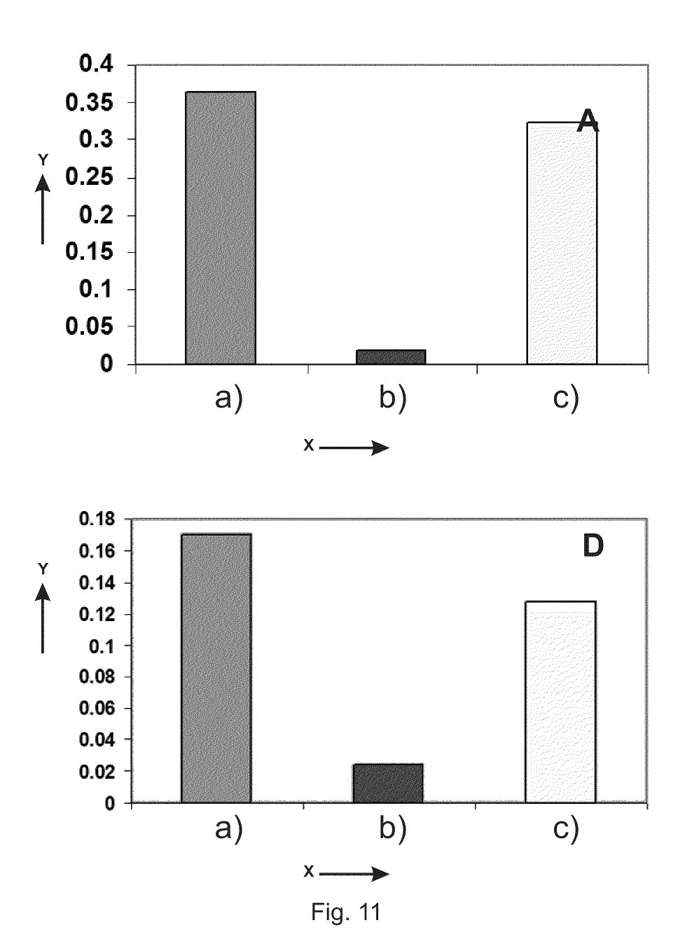

Release kinetics of the cyanine dye: The cyanine dye 9 (cypate) was post loaded in ORMOSIL nanoparticles as described above. FIG. 11 A shows that the dye was retained by the nanoparticles with high efficiency. To study the release kinetics, nanoparticles post-loaded with the cyanine dye were incubated in 1% BSA for 6 h and then spin filtered as described above. Results indicated that most of the dye was firmly embedded in nanoparticles.

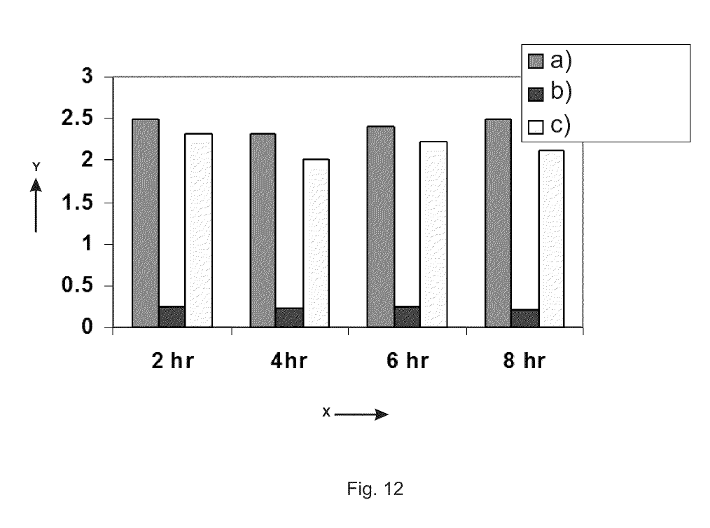

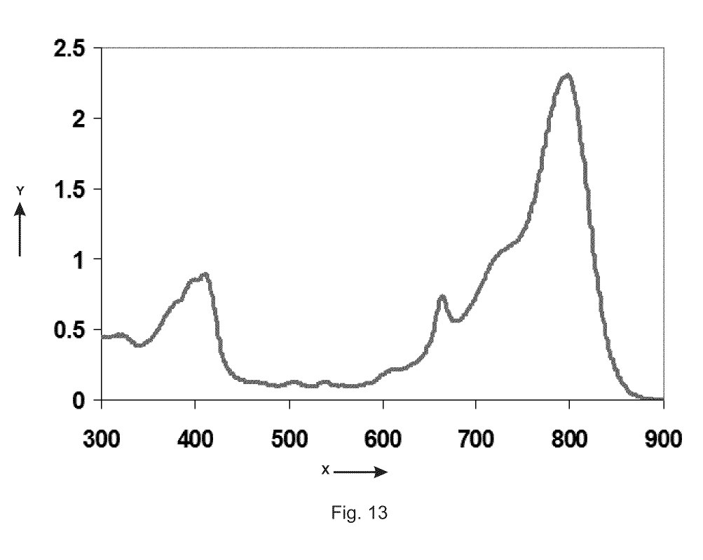

Post-loading of the cyanine dye in HPPH-conjugated ORMOSIL nanoparticles: In another set of experiment, the HPPH-conjugated nanoparticles were prepared by following our own methodology (U.S. patent application submitted) and the cyanine dye (a IR fluorescent agent) was post-loaded. The release of the cyanine dye at various time points was determined by following the methodology discussed above and the concentrations of both the chromophores (HPPH and the cyanine dye) in the filtrates and in the nanoparticles were determined by fluorescence spectroscopy (FIG. 12). The absorption spectrum shown in FIG. 13 clearly demonstrates the presence of both the chromophores (HPPH, 660 nm and the cyanine dye, 797 nm) in ORMOSIL nanoparticles.

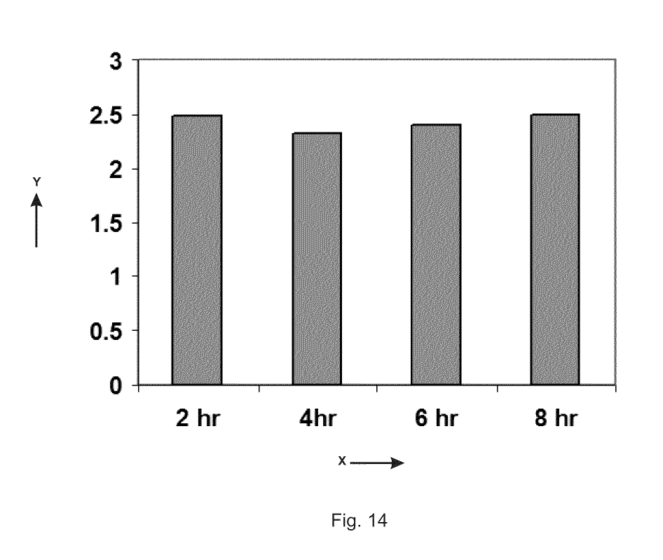

From FIG. 14 it can be seen that even on excessive washing of the nanoparticles at a high spin-speed only a small percentage of the cyanine dye was released It was observed that under the conditions used for the encapsulation of the cyanine dye, the dye starts decomposing in a short period (<2 h). In contrast with encapsulation process, under post-loading, the cyanine dyes were remarkably stable and did not show any significant change in its fluorescence intensity.

### Comparative Release Kinetics of Encapsulated and Post-Loaded HPPH and the Post Loaded Cationic Photosensitizer.

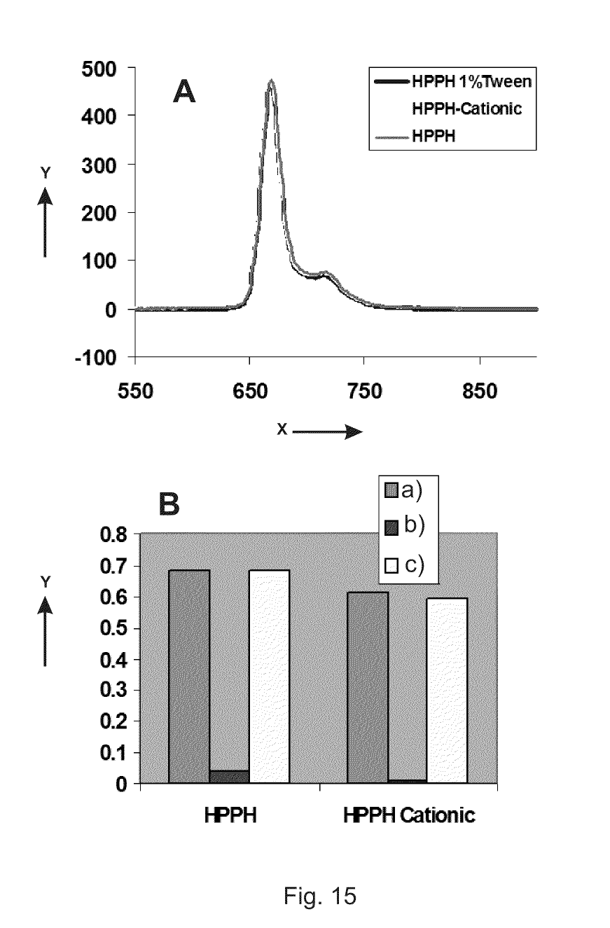

In order to investigate the effect of charge in post-loading, HPPH and the cationic photosensitizer 8 were individually post-loaded to ORMOSIL nanoparticles and the release kinetics was measured by following the methodology as discussed above. The results summarized in FIG. 15 clearly indicate that both HPPH 7 and the corresponding cationic analog 8 show their high concentrations in ORMOSIL after post-loading.

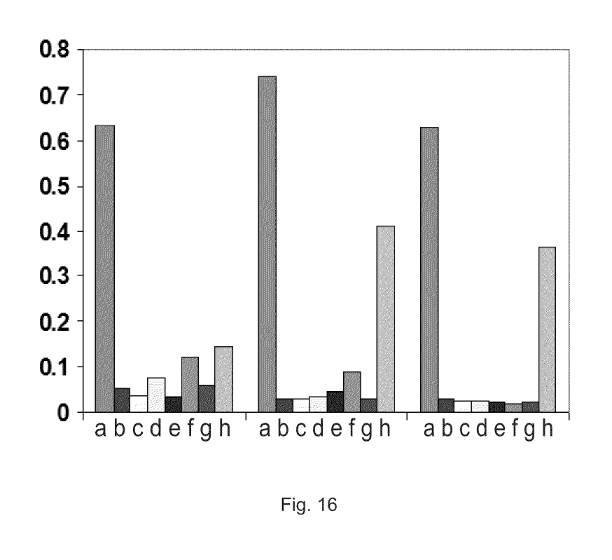

Our next objective was to investigate the superiority of post-loading over encapsulating technique in drug delivery. The results summarized in FIG. 16 clearly show a significant difference in the release kinetics of encapsulated over post-loading photosensitizers in ORMOSIL nanoparticles. These results suggest that compared to the encapsulation process the post loading technique could be more reliable for a high “pay-load” of the desired tumor imaging and/r therapeutic agents in tumors. The post-loading technique also provides an opportunity for the development of watersoluble “Multimodality” agents.

### In Vitro Activity:

### In Vitro and In Vivo Photosensitizing Efficacy:

The photosensitizing activity of the encapsulated or post-loaded photosensitizers were determined in Colon-26 cell lines. The cells were grown in -MEM with 10% fetal calf serum, L-glutamine, penicillin and streptomycin. Cells were maintained in 5% CO₂, 95% air and 100% humidity. Cells were plated in 96-well plates at a density of 5×10³ cells well in complete medium. After an overnight incubation at 7′C, the photosensitizers were added at varying concentrations and incubated at 37° C. for 3 or 24 hr in the dark. Prior to light treatment the cells were replaced with drug-free complete medium. Cells were then illuminated with a white light of 3.2 mW/cm² for 0.5 or 1.0 J/cm². After PDT, the cells were incubated for 48 hr at 37′C in the dark. Following the 48 hr incubation, 10 l of 4.0 mg/ml solution of 3-[4,5-dimethylthiazol-2-yl]-2,5-diphenyltetrazoliumbromide (MTT) (Sigma, St. Louis, Mo.) dissolved in PBS was added to each well. After 4 hr incubation at 37′C unreacted MTT and medium were removed and 100 μl DMSO was added to solubilize the formazan crystals. The 96-well plate was read on a microtiter plate reader (Miles Inc. Titertek Multiscan Plus MK II) at an absorbance of 560 nm. The results were plotted as percent survival of the corresponding dark (drug no light) control for each compound tested after subtracting medium only control absorbance. Drug dose-cell survival curves were generated (using Microcal™ Origin 6.0) and LD₅₀ value was measured on the curve based on Gaussian and Sigmoidal fitness. Each data point represents the mean from 3 separate experiments with 6 replicates at each dose, and the standard errors were less than 10%.

### In Vitro Activity of HPPH, HPPH-Encapsulated and HPPH-Postloaded ORMOSIL Nanoparticles:

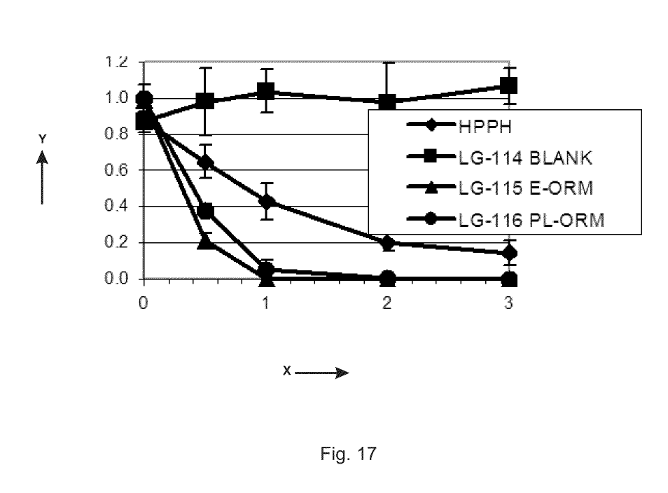

The photosensitizing efficacy of these analogs was determined in at equimolar concentrations (1 μM) in colon-26 cells at 4 h incubation at variable light doses. As can be seen from FIG. 17, both HPPH encapsulated and post-loaded formulations exhibited similar efficacy. Interestingly these formulations were found to be more effective than the HPPH alone. The higher activity of encapsulated and post-loaded HPPH over HPPH (1% Tween 80) could be due to higher cell-uptake and these studies are currently in progress.

### In Vitro Activity of Post-Loaded Purpurinimide 1 and Bacteriopurpurin-Imide 2:

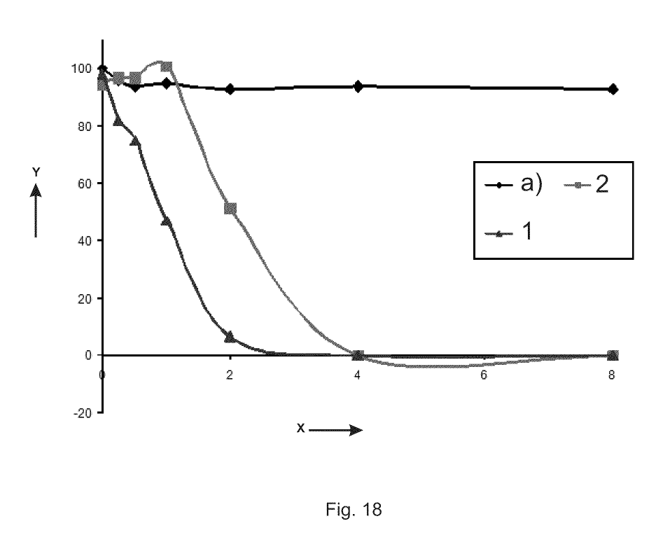

The post-loaded purpurinimide 1 (700 nm) and bacteriopurpurinimide 2 (792 nm) were evaluated for in vitro photosensitizing efficacy in Colon-26 cells by following the procedure discussed above except for purpurinimide 1 the cells were exposed to a laser light of 700 nm and for bacteriopurpurinimide 2, they were exposed at 792 nm (the long-wavelength absorption). The results are summarized in FIG. 18. As can be seen, both photosensitizers were quite effective in vitro.

### In Vivo Photosensitizing Efficacy:

The BALB/c mice were intradermally injected with 2×10⁵ Colon-26 cells in 30 ml HBSS without Ca²⁺ and Mg²⁺ on the flank and tumors were grown until they reached 4-5 mm in diameter. The day before laser light treatment, all hair was removed from the inoculation site and the mice were injected intravenously with varying photosensitizer concentrations. At 24 hours post-injection, the mice were restrained without anesthesia in plastic holders and then treated with laser light from a dye laser tuned to emit drug-activating wavelengths (705 nm for purpurinimide, the in vivo absorption of the drug) and 796 nm for bacteriopurpurinimide) at a dose of 135 J/cm². The mice were observed daily for signs of weight loss, necrotic scabbing, or tumor re-growth. If tumor growth appeared, the tumors were measured using two orthogonal measurements L and W (perpendicular to L) and the volumes were calculated using the formula V=(L×W²)/2 and recorded. Mice were considered cured if there was no sign of tumor re-growth by day 90 (2160 h) post-PDT treatment.

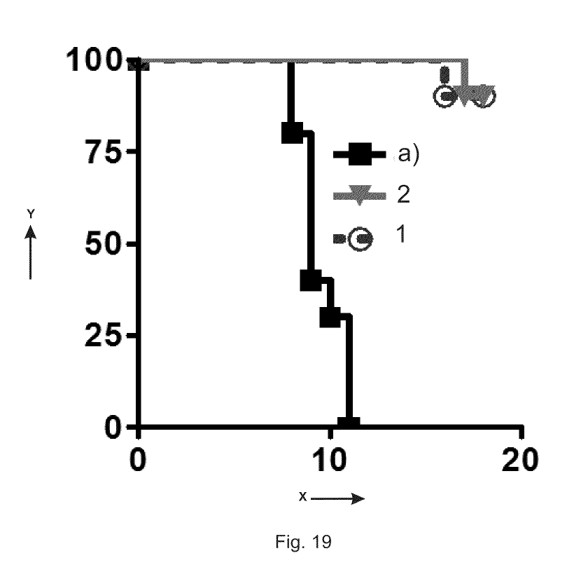

From the results summarized in FIG. 19, it can be seen that both compounds at a dose of 1.0 μmole/kg are quite effective. Further studies to determine the long-term tumor response are currently in progress.

### Fluorescence Imaging:

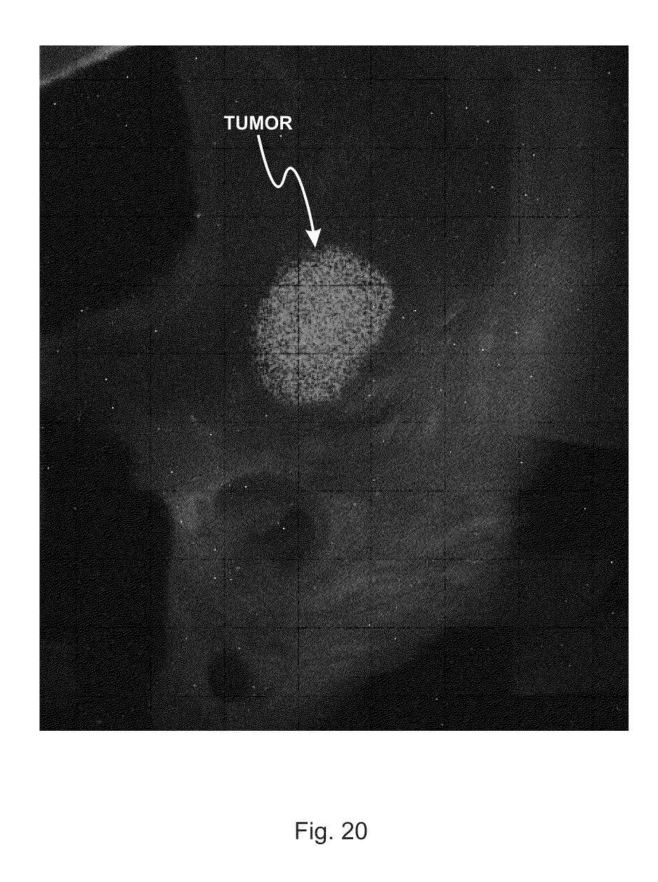

The delivery of the photosensitizer (e.g. HPPH 7) in tumors was also confirmed by fluorescence imaging. In a typical experiments BALB/c mice bearing Colon-26 tumors were injected with HPPH-post-loaded ORMOSIL nanoparticles (drug conc. 0.47 mmol/Kg) and the tumors were detected by fluorescence imaging (λ_{Ex}: 530 and λ_{Em}:670 nm). The fluorescence image (false colors) shown in FIG. 20 clearly show a high concentration of the drug in the tumor than the surrounding muscle. The optimal imaging parameters (drug concentration, imaging time points etc. etc.) were not optimized. Further studies with various cyanine dyes [longer wavelength absorbing chromophores (750 to 800 nm)] post-loaded ORMOSIL nanoparticles are currently in progress.

In accordance with this invention ORMOSIL-based nanoparticles for tumor imaging (fluorescence, PET, SPECT) and therapy are developed including methods for using them.

The invention further includes a method for forming nanoparticles containing post loaded photosensitizer.

A specific method for forming such nanoparticles includes the steps of:

a) forming a uniform medium comprising from about 70 to about 80 weight percent of a lower alcohol selected from isopropanol, n-butanol, isobutanol and n-pentanol, from about 20 to about 30 weight percent of DMSO, from about 2 to about 3 percent water and from about 0.025 to about 0.15 percent of sufficient surfactant to maintain a dispersion;

b) uniformly incorporating one or more siloxanes, as above described wherein the amount of siloxanes or mixture of siloxanes is about the maximum permitted for stability;

c) adding sufficient reactive basic compound to form nanoparticles;

d) dialyzing the nanoparticles through a membrane having a pore size of from about 0.1 to about 0.3 μM to obtain blank nanoparticles;

e) mixing photosensitizer in DMSO solution with blank nanoparticle from step d) for a time to obtain post loaded photosensitizer nanoparticles; and

f) dialyzing the resulting mixture against distilled water using a membrane having a of cut-off pore size of 12-14 kD for the removal of residual DMSO and any loosely bound photosensitizer to obtain purified post loaded nanoparticles.

To separate the Tween-80 micelles (and any loosely bound PS) from the nanoparticles, the dialyzed dispersions were filtered and residue was washed three times with water through a microfuge membrane-filter (NANOSEP 100K OMEGA, Pall Corporation, USA) by centrifuging at 9,000 rpm for 30 minutes (spin-filtration). Tween-80 micelles and loosely bound PS molecules flow-through this membrane and are collected in the lower tube (flow-fraction), while nanoparticles get embedded in the membrane and can be subsequently extracted by adding water and sonicating/vortexing (membrane-fraction). This accomplished by providing reactive intermediate structures on the nanoparticle, either by providing them on the nanoparticle precursor or by adding them subsequent to nanoparticle formation.

The surfactant used in the method is usually a polyoxyethylene sorbitan monooleate or sodium dioctyl sulfosuccinate and the silane usually includes: vinyltrimethoxysilane, vinyltriethoxysilane, vinylytriacetosilane, γ-glycidoxypropyltrimethoxysilane, γ-methacryloxy-propyltrimethoxysilane, γ-aminopropyltrimethoxysilane, γ-aminopropyltriethoxysilane, γ-mercaptopropyltrimethoxysilane, γ-3,4-epoxycyclohexyltrimethoxysilane and phenyltrimethoxysilane.

The siloxane is preferably vinyltriethoxysilane or phenyltrimethoxysilane and the basic compound is usually ammonia or 3-aminopropylethoxysilane. It should; however be understood that essentially any base may be used provided that it if it is a strong base, e.g. an alkali hydroxide, it is sufficiently diluted.

Preferred photosensitizers are preferentially absorbed or adsorbed by cells that require destruction or significant alteration, e.g. cells of hyperproliferative tissue such as tumor cells, hypervascularization such as found in macular degeneration and hyperepidermal debilitating skin diseases. Selectivity can be further enhanced by incorporating with nanoparticles in accordance with the present invention, targeting agents such as an monoclonal antibodies, integrin-antagonists or carbohydrates which have high affinity for target tissue (mainly cancer).

Preferred photosensitizers are tetrapyrrole-based compounds related to porphyrins, chlorins, bacteriochlorins, benzochlorins, benzoporphyrin derivatives, pheophorbides including pyropheophorbides, and phthalocyanines and, naphthanocyanines with and without fused ring systems and derivatives of all the above.

A desirable photosensitizer for many applications is a tumor avid tetrapyrollic photosensitizer, that may be complexed with an element X where X is a metal selected from the group consisting of Zn, In, Ga, Al, or Cu or a radioisotope labeled moiety wherein the radioisotope is selected from the group consisting of ¹¹C, ¹⁸F, ⁶⁴Cu, ¹²⁴I, ⁹⁹Tc, ¹¹¹In and GdIII that may be used in a method for diagnosing, imaging and/or treating hyperproliferative tissue such as tumors and other uncontrolled growth tissues such as found in macular degeneration.

In the case of the presence of a tetrapyrollic photosensitizer, the photosensitizer usually has the structural formula:

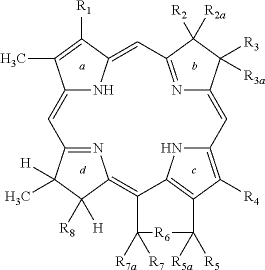

and its complexes with X where:

### R₁ is —CH═CH₂, —CH₂CH₃, —CHO, —COOH, or

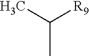 [0121] where R₉═—OR₁₀ where R₁₀ is lower alkyl of 1 through 8 carbon atoms, —(CH₂—O)ₙCH₃, —(CH₂)₂CO₂CH₃, —(CH₂)₂CONHphenyleneCH₂DTPA, —CH₂CH₂CONH(CONHphenyleneCH₂DTPA)₂, —CH₂R₁₁ or

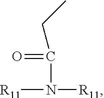

or a fluorescent dye moiety; R₂, R₂ₐ, R₃, R₃ₐ, R₄, R₅, R₅ₐ, R₇, and R₇ₐ are independently hydrogen, lower alkyl or substituted lower alkyl or two R₂, R₂ₐ, R₃, R₃ₐ, R₅, R₅ₐ, R₇, and R₇ₐ groups on adjacent carbon atoms may be taken together to form a covalent bond or two R₂, R₂ₐ, R₃, R₃ₐ, R₅, R₅ₐ, R₇, and R₇ₐ groups on the same carbon atom may form a double bond to a divalent pendant group; R₂ and R₃ may together form a 5 or 6 membered heterocyclic ring containing oxygen, nitrogen or sulfur; R₆ is —CH₂—, —NR₁₂— or a covalent bond; R₈ is —(CH₂)₂CO₂CH₃, —(CH₂)₂CONHphenyleneCH₂DTPA, —CH₂CH₂CONH(CONHphenyleneCH₂DTPA)₂, —CH₂R₁₁ or

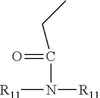

where R₁₁ is —CH₂CONH-RGD-Phe-Lys, —CH₂NHCO-RGD-Phe-Lys, a fluorescent dye moiety, or —CH₂CONHCH₂CH₂SO₂NHCH(CO₂)CH₂NHCO-PhenylOCH₂CH₂NHcycloCNH(CH₂)₃N; R₁₂ is hydrogen, lower alkyl or substituted lower alkyl; and X complexes thereof; where X is a metal selected from the group consisting of Zn, In, Ga, Al, or Cu or a radioisotope labeled moiety wherein the radioisotope is selected from the group consisting of ¹¹C, ¹⁸F, ⁶⁴Cu, ¹²⁴I, ⁹⁹TC, ¹¹¹In and GdIII.

The complexes with X are readily made simply by heating the compound with a salt of X such as a chloride.

The complex will form as a chelate of a -DTPA moiety, when present, or within the tetrapyrollic structure between the nitrogen atoms of the amine structure or both. Examples of such structures are:

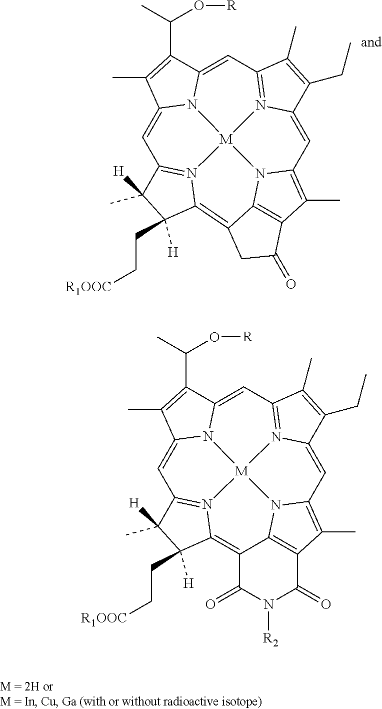

Where X=M

What is claimed is:

1. A nanoparticle comprising a polysiloxane base having an exterior surface and having a photosensitizer at least partly exposed at its exterior surface, said photosensitizer being secured to the exterior surface by loading the photosensitizer onto the surface after formation of the polysiloxane base of the nanoparticle.

2. The nanoparticle of claim 1 where the nano particle comprises tumor targeting moieties.

3. The nanoparticle of claim 1 where nanoparticle comprises a post loaded cyanine dye.

4. The nanoparticle of claim 1 where the nanoparticle comprises post loaded moieties providing at least two of tumor specificity, photodynamic properties and imaging capabilities.

5. The nanoparticle of claim 1 where at least a portion of the photosensitizer is tagged with a radioisotope.

6. The nanoparticle of claim 1 where the nanoparticle comprises post loaded moieties providing multifunctional selected from the group consisting of photosensitivity, PET detectability, fluorescence and target specificity.

7. A nanoparticle having the structural formula: 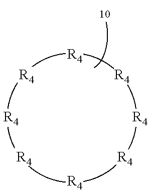
where the ring represents a siloxane matrix,
R₄ is (R₁)ₙ-(R₂)ₙ where R₁ and R₂ are independently at each occurrence a labeled photosensitizer (IP), unlabeled photosensitizer (P), cyanine dye, SPECT imaging agent, PET imaging agent, MR imaging agent, radionucleotide imaging agent or fluorescent imaging agent, cyanine dye, biotargeting moiety, linked targeting agent RGD, F3 peptide, carbohydrate or folic acid at least partially available at a surface of the siloxane matrix, and
n is 0 or 1; provided that, at least one n is 1 and the compound contains at least one labeled or unlabeled photosensitizer.

8. The nanoparticle of claim 7 where at least one R₁ or R₂ group is a tetrapyrollic photosensitizer.

9. The nanoparticle of claim 8 where the tetrapyrollic photosensitizer is a porphyrin, chlorin, bacteriochlorin, benzochlorin, benzoporphyrin, or pheophorbides or derivatives thereof.

10. The nanoparticle of claim 9 where the tetrapyrollic photosensitizer is a pyropheophorbide.

11. The nanoparticle of claim 7 where a plurality of R₁ groups are located at peripheral positions on the nanoparticle and a plurality of R₂ groups are imaging agents located at peripheral positions on the nanoparticle.

12. The nanoparticle of claim 7 where the biotargeting moiety comprises a linked targeting agent selected from the group consisting of RGD, F3 peptide, carbohydrate, folic acid, or antibody specific for a tumor ligand.

13. A method for forming a nanoparticle of claim 1 including the steps of:
a) forming a uniform medium comprising from about 70 to about 80 weight percent of a lower alcohol selected from isopropanol, n-butanol, isobutanol and n-pentanol, from about 20 to about 30 weight percent of DMSO, from about 2 to about 3 percent water and from about 0.025 to about 0.15 percent of sufficient surfactant to maintain a dispersion;
b) uniformly incorporating one or more siloxanes, as above described wherein the amount of siloxanes or mixture of siloxanes is about the maximum permitted for stability;
c) adding sufficient reactive basic compound to form nanoparticles;
d) dialyzing the nanoparticles through a membrane having a pore size of from about 0.1 to about 0.3 μM to obtain blank nanoparticles;
e) mixing photosensitizer in DMSO solution with blank nanoparticle from step d) for a time to obtain post loaded photosensitizer nanoparticles; and
f) dialyzing the resulting mixture against distilled water using a membrane having a of cut-off pore size of 12-14 kD for the removal of residual DMSO and any loosely bound photosensitizer to obtain purified postloaded nanoparticles.

14. The method of claim 13 where the siloxane is condensed vinyltriethoxysilane.
